(19) United States
(12) Patent Application Publication
(10) Pub. No.: US 2001/0034459 A1
(43) Pub. Date: Oct. 25, 2001

(54) Process for preparing 4'-trifluoromethyl-2-methylbiphenyl and 4'-trifluoromethyl-biphenyl-2-carboxylic acid from o-tolylmetallates

(76) Inventors: Andreas Meudt, Florsheim-Weilbach (DE); Stefan Scherer, Buttelborn (DE); Antje Norenberg, Buttelborn (DE); Peter Koch, Frankfurt am Main (DE); Steffen Haber, Landau/Pfalz (DE)

Correspondence Address: Clariant Corporation, Industrial Property Department, 4331 Chesapeake Drive, Charlotte, NC, 28216, US

(21) Appl. No.: 09/742,740

(22) Filed: Dec. 21, 2000

(30) Foreign Application Priority Data
Dec. 29, 1999 (DE) 19963562.5

(51) Int. Cl. C07C 51/16; C07C 51/255; C07C 22/04

(52) U.S. Cl. 562/409; 562/410; 562/412; 562/416; 570/140; 570/141; 570/142

(57) ABSTRACT

The invention relates to a process for preparing a compound of the formula (I)

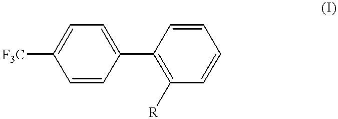

in which R is methyl or carboxyl, which comprises coupling an ortho-tolylmetallate of the formula (II) with a compound of the formula (III)

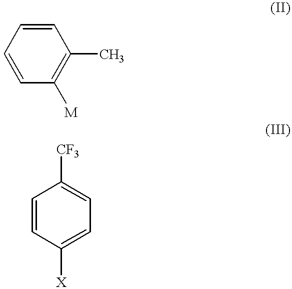

in which

M is —MgF, —MgCl, —MgBr, —Mgl, —Li, —ZnF, —ZnCl, —ZnBr or —Znl and

X is F, Cl, Br, I, N₂⁺, straight-chain or branched (C₁-C₂₀)-alkoxy, arylsulfonate or alkylsulfonate

in the presence of an Ni, Pd or platinum metal catalyst to give a compound of the formula (I) where R is CH₃ and, if appropriate, oxidizing the compound of the formula (I) where R is CH₃ to give the compound of the formula (I) where R is carboxyl.

### BACKGROUND OF THE INVENTION

The invention relates to a novel process for preparing compounds of the formula (I)

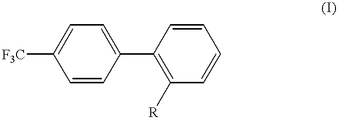

in which R is methyl or carboxyl.

Trifluoromethylbiphenyl-2-carboxylic acid is an important intermediate in the synthesis of active compounds in the pharmaceutical industry. Owing to the great importance, a number of syntheses have been described in the literature; however, all of them are characterized by specific economical and technical disadvantages.

U.S. Pat. No. 4,578,522 and EP-A-0 059 983 describe the coupling of p-trifluoromethylphenylmagnesium bromide with dimethyloxazolineanisole and subsequent acid hydrolysis of the oxazoline radical; here, the overall yield is 28.4% (coupling 33%; hydrolysis 86%). In addition to the unsatisfactory yields, the use of the oxazoline protective group for the acid function is disadvantageous here.

In a second process (U.S. Pat. No. 4,578,522, EP-A-0 059 983), the Grignard compound of o-bromobenzaldehyde dimethyl acetal is coupled, under palladium catalysis, with p-iodobenzotrifluoride. Following chromatography, the yield is 86%, based on the iodoaromatic compound. In a second step, the aldehyde is oxidized to the carboxylic acid using KMnO₄ (yield 85%). Here, it is in particular the use of bromo- or iodoaromatic compounds, which are difficult to obtain, which is disadvantageous.

A third variant (J. Org. Chem. 1977, 42,1821; J. Labelled Compd. and Radiopharm. 1992, 21, 2011) uses phenyidimethyloxazoline as starting material, which is selectively deprotonated with n-butyllithium, transmetallated with zinc chloride and, under Pd catalysis, coupled with p-iodobenzotrifluoride. Acidic cleavage of the oxazoline gives the target molecule.

In a further process, p-chlorobenzotrifluoride is, under nickel catalysis, coupled with the ortho-zinc compound of phenyldimethyloxazoline (Org. Prep. Proced. Int. 1995, 27 (3), 367). Here, the target compound is obtained in a yield of 64%, based on

p-chlorobenzotrifluoride.

In these two processes, the significance of the relatively good yields is reduced by a complicated and costly synthesis. The use of the oxazoline protective group and the associated atom-economical and technical disadvantages are common to all of the processes described.

The preparation of 4′-trifluoromethyl-2-methylbiphenyl by crosscoupling has been described by A. Indolese, Tetrahedron Lett. 38, 20, 1997, 3513-3516. The coupling of o-tolylboronic acid with p-chlorobenzotrifluoride under catalysis with NiCl₂(dppf) in dioxane gives a 4′-trifluoromethyl-2-methylbiphenyl yield of only 26%.

### SUMMARY OF THE INVENTION

It was the object of the present invention to provide a process for preparing 4′-trifluoromethyl-2-methylbiphenyl and 4′-trifluoromethylbiphenyl-2-carboxylic acid which can be carried out on an industrial scale and does not have the abovementioned disadvantages and, in particular, avoids using expensive protective groups, such as the oxazoline protective group. Furthermore, the process to be developed should, if possible, not entail any technically complicated purification procedures, such as recrystallization or chromatography, and afford the product in a purity sufficiently high for the preparation of pharmaceutically active compounds.

### DESCRIPTION OF THE PREFERRED EMBODIMENTS

Surprisingly, it has been found that 4′-trifluoromethylbiphenyl-2-carboxylic acid can be obtained in a simple technical process, in good yields and in high purity by a two-step process in which a) o-tolylmetallates of the formula (II) are coupled with trifluoromethylbenzenes of the formula (III) carrying a suitable leaving group X in the 4-position and b) oxidizing the resulting 4′-trifluoromethyl-2-methylbiphenyl to the corresponding carboxylic acid using suitable oxidizing agents.

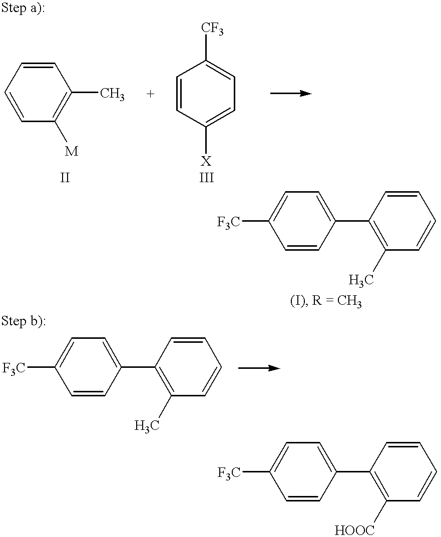

The present invention provides a process for preparing a compound of the formula (I)

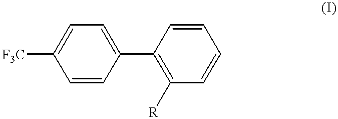

in which R is methyl or carboxyl, which comprises coupling an ortho-tolylmetallate of the formula (II) with a compound of the formula (III)

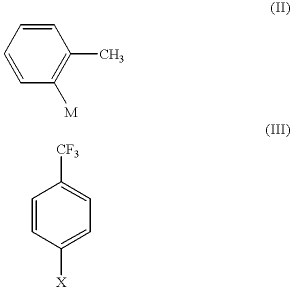

in which

M is —MgF, —MgCl, —MgBr, —Mgl, —Li, —ZnF, —ZnCl, —ZnBr or —Znl and

X is F, Cl, Br, I, N₂⁺, straight-chain or branched (C₁-C₂₀)-alkoxy, arylsulfonate or alkylsulfonate

in the presence of an Ni, Pd or platinum metal catalyst to give a compound of the formula (I) where R is CH₃ and, if appropriate, oxidizing the compound of the formula (I) where R is CH₃ to give the compound of the formula (I) where R is carboxyl.

Preferred o-tolylmetallates are o-tolylmagnesium chloride and o-tolylmagnesium bromide.

Preferred compounds of the formula (III) are those in which X is chlorine, bromine, triflate, tosylate, nonaflate, mesylate, and particular preference is given to

4-chlorotrifluoromethylbenzene.

Suitable catalysts for the coupling reactions have been described in the literature and are familiar to the person skilled in the art. Preference is given here to salts, complexes or the metallic form of nickel, palladium or another platinum metal, such as, for example, Rh and Pt.

Particular preference is given to salts or complexes of nickel or palladium and also to metallic forms, if appropriate on a suitable support, for example Pd on C or on BaSO₄, very particularly preferably PdCl₂(dppf), PdCl₂(PPh₃)₂, PdCl₂(dppe), PdCl₂(dppp), PdCl₂(dppb), Pd(PPh₃)₄, Pd(OAc)₂, PdCl₂, PdBr₂ or NiCl₂(PPh₃)₂, in which “dppf” is 1,2-bis-(diphenylphosphino)ferrocene, “dppe” is 1,2-bis(diphenylphosphino)-ethane, “dppp” is 1,2-bis(diphenylphosphino)propane, “dppb” is 1,2-bis-(diphenylphosphino)-butane, “Ph” is phenyl and “Ac” is acetyl. The amount of catalyst can be from 0.00001 to 50 mol %, preferably from 10⁻⁴ to 10 mol %, based on the compound of the formula (III).

The coupling is advantageously carried out in solvents, such as, for example, aliphatic or aromatic ethers, aromatic or aliphatic hydrocarbons, halogenated aromatic or aliphatic hydrocarbons or mixtures of the solvents mentioned, preferably in THF or THF/toluene mixtures.

Advantageous reaction temperatures are, depending on the activity of the catalyst and the substrates, from 0° C. to 180° C., preferably from 30° C. to 150° C., particularly preferably from 50 to 100° C.

The molar ratios of the compound of the formula (II) to (III) are advantageously (II):(III) from 0.85:1 to 1.2:1.

Under the conditions according to the invention, the formation of dimers, i.e. 2,2′-dimethylbiphenyl and 4,4′-bis(trifluoromethyl)biphenyl by homocoupling is surprisingly prevented virtually completely.

It has been found to be advantageous to meter the solution or suspension of the o-tolylmetallate into a solution of the substituted trifluoromethylbenzene and the catalyst. However, it is also possible to meter the trifluoromethylbenzene or a solution of the trifluoromethylbenzene in a solvent into the solution or suspension of the o-tolylmetallate, or to add a mixture of the reactants dropwise to the solution of the catalyst.

Work-up is advantageously carried out by introducing, after the reaction has ended, the reaction solution into water or an aqueous solution of a suitable acid, preferably hydrochloric acid or sulphuric acid, temperatures from 0 to 80° C. and acid concentrations from 0 to 35% by weight being preferred. The 4′-trifluoromethyl-2-methylbiphenyl dissolved in the organic phase can be obtained in very high purity by distillation.

The oxidation of 4′-trifluoromethyl-2-methylbiphenyl can be effected by oxidizing agents such as nitric acid, permanganate, chromium(VI) compounds, oxygen or air. Preference is given here to potassium permanganate, sodium permanganate or air and in particular to air.

The oxidation with potassium permanganate can be carried out either in aqueous solution or in nonaqueous medium. In aqueous solution, the presence of suitable phase-transfer catalysts, for example tetraalkylammonium salts, tetraphenylphosphonium salts or crown ethers promotes good yields. Work-up is carried out by filtering off the manganese dioxide formed, acidification and filtering off the 4′-trifluoromethylbiphenyl-2-carboxylic acid.

Oxidation with air is carried out in aliphatic carboxylic acids, if appropriate in a mixture with water. Preference is given to using acetic acid, and particular preference is given to a mixture of acetic acid and water. The catalysts used are heavy metal salts, for example mixtures of cobalt and manganese salts, in particular the bromides. The heavy metal salts are employed in amounts of in each case from 0.01 to 5.0 mol %, preferably from 0.1 to 4.0 mol %, particularly preferably from 1.0 to 2.0 mol %, based on the 4′-trifluoromethyl-2-methylbiphenyl. The ratio of cobalt and manganese salts can be varied within wide limits and can, for example, be from 1:5 to 5:1. The two salts are preferably employed in equimolar amounts. The reaction is carried out at temperatures from 100 to 200° C., preferably from 120 to 180° C. and particularly preferably from 150 to 170° C. The pressure is from atmospheric pressure to 50 bar. Work-up is carried out by cooling and, if required, concentration of the reaction mixture, filtration, washing and drying of the resulting precipitated carboxylic acid. In the present case, a mixture of the desired 4′-trifluoromethylbiphenyl-2-carboxylic acid and the fluorenone of the formula (V)

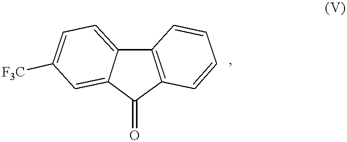

formed from the former under the reaction conditions, is obtained. From this mixture, the desired biphenylcarboxylic acid can be isolated in pure form in a simple manner by extraction with alkali and subsequent precipitation with mineral acids.

### EXAMPLES

Examples of the Preparation of 4′-trifluoromethyl-2-methylbiphenyl

### Example 1

Coupling of o-tolylmagnesium chloride with p-trifluoromethylchlorobenzene

Over a period of 2 hours, a 26% by weight strength solution of o-tolylmagnesium chloride (5.0 mol) in THF was metered into a boiling solution of 993 g of p-trifluoromethylchlorobenzene (5.5 mol) and palladium(II) chloride(dppf) (4.1 g; 0.1 mol %) in 1316 g of THF. After a reaction time of six hours at the boiling point of the mixture (73° C.), aqueous work-up was carried out using 600 ml of 0.1% by weight strength sulfuric acid. The product was obtainable in pure form by fractional vacuum distillation (105° C./8 mbar), the yield was 940 g (94.7%).

Reduction of the amount of catalyst to 100 mg (0.0023 mol %) and simultaneous increase of the reaction time to 11 hours resulted in a similar yield.

### Example 2

Coupling of o-tolylmagnesium bromide with p-trifluoromethylchlorobenzene

The coupling was carried out analogously to the reaction described in Example 1 (0.01 mol % of PdCl₂(dppf); 6 hours). The yield was 93.2%.

### Example 3

Coupling of o-tolylmagnesium chloride with p-trifluoromethylbromobenzene

The coupling was carried out analogously to the reaction described in Example 1 (0.001 mol % of PdCl₂(dppf)). The reaction had ended after only 1 hour; the yield was 96.1%.

### Example 4

Coupling of o-tolyllithium with p-trifluoromethylchlorobenzene

4′-trifluoromethyl-2-methylbiphenyl was obtained in a yield of 78% by reacting o-tolyllithium in toluene with one equivalent of p-trifluoromethylchlorobenzene and 0.1 mol % of PdCl₂(PPh₃)₂ at 50° C./0.5 h.

### Example 5

Coupling of o-tolylmagnesium chloride with p-trifluoromethylphenyl triflate

4′-Trifluoromethyl-2-methylbiphenyl was obtained in a yield of 95.5% by reacting a 26% by weight strength solution of o-tolylmagnesium chloride in THF with one equivalent of p-trifluoromethylphenyl triflate (obtainable from p-trifluoromethylphenol and trifluoromethanesulfonic anhydride or trifluoromethanesulfonyl chloride/triethylamineldichloromethane/0. 1% DMAP) and 0.1 mol % of PdCl₂(dppf) at 50° C./0.5 h.

Examples of the Preparation of 4′-trifluoromethylbiphenyl-2-carboxylic acid

### Example 6

Oxidation with Potassium Permanganate in Aqueous Solution

At room temperature, 1.0 g of anhydrous sodium carbonate and 1.13 g of 4′-trifluoromethyl-2-methylbiphenyl were added to 75 ml of a 2% by weight strength aqueous potassium permanganate solution. The mixture was stirred at 100° C. for

1.5 hours, and 4′-trifluoromethylbiphenyl-2-carboxylic acid was obtainable in a yield of 21% by filtering off the precipitated manganese dioxide, acidification and filtering off the precipitated product.

### Example 7

Oxidation with Potassium Permanganate in Aqueous Solution Using A Phase-Transfer Catalyst

At room temperature, 1.0 g of anhydrous sodium carbonate, 0.2 g of benzyltributylammonium chloride and 1.13 g of 4′-trifluoromethyl-2-methylbiphenyl were added to 75 ml of a 2% by weight strength aqueous potassium permanganate solution. The mixture was stirred at 100° C. for 1.5 hours, after which

4′-trifluoromethylbiphenyl-2-carboxylic acid was obtainable in a yield of 76% by filtering off the precipitated manganese dioxide, acidification and filtering off the precipitated product.

### Example 8

Oxidation with Air

In a 3.51 autoclave, 177.2 g (0.75 mol) of 2-methyl-4′-trifluoromethylbiphenyl, 1511.2 g of acetic acid, 3.74 g of (15.0 mmol) of cobalt(II) acetate tetrahydrate, 3.68 g (15,0 mmol) of manganese(II) acetate tetrahydrate and 3.09 g (30.0 mmol) of sodium bromide are initially charged. The autoclave is rendered inert using nitrogen and heated to 160-165° C. When this temperature is reached, about 450 l of air/h are introduced, and an internal pressure of 16-18 bar is maintained by a pressure-regulating system. Air is introduced for about 30-40 minutes, the autoclave is then once more rendered inert by applying nitrogen and the mixture is cooled. The greenish solution (which turns orange-red on cooling) is discharged from the autoclave and, using a rotary evaporator, concentrated to 370 g. The viscous suspension is filtered off using nutsch filter, and the crystals are washed 3 times with in each case 50 g of 75% strength acetic acid. This gives 155 g of

4′-trifluoromethylbiphenyl-2-carboxylic acid (77.6% of theory) which is still contaminated with the fluorenone of the formula (V).

### Example 9 Oxidation with Air

In a 3.51 autoclave, 177.2 g (0.75 mol) of 2-methyl-4′-trifluoromethylbiphenyl, 1350 g of acetic acid, 150 g of water, 3.74 g of (15.0 mmol) of cobalt(II) acetate tetrahydrate, 3.68 g (15,0 mmol) of manganese(II) acetate tetrahydrate and 3.09 g (30.0 mmol) of sodium bromide are initially charged. The autoclave is rendered inert using nitrogen and heated to 160-165° C. When this temperature is reached, about 450 l of air/h are introduced, and an internal pressure of 16-18 bar is maintained by a pressure-regulating system. Air is introduced for about 30-40 minutes, the autoclave is then once more rendered inert by applying nitrogen and the mixture is cooled. The greenish solution (which turns orange-red on cooling) is discharged from the autoclave and, using a rotary evaporator, concentrated to 350 g. The viscous suspension is filtered off using nutsch filter, and the crystals are washed 3 times with in each case 50 g of 75% strength acetic acid. This gives 180 g of

4′-trifluoromethylbiphenyl-2-carboxylic acid (90.2% of theory) which is still contaminated with the fluorenone of the formula (V).

### Example 10

Purification of the Products from the Oxidation with Air

136.5 g of the crude product from Example 8 or 9 are introduced into a mixture of 100 g of 33% strength aqueous sodium hydroxide solution and 900 g of water, and the mixture is stirred for 30 min. The undissolved solid is filtered off and the residue is washed with water and dried.

2-Trifluoromethyl-9-fluorenone

| | |
|---|---|
| Yield: | 15.5 g (yellow crystals) |
| | 11.4% of the quantity employed |
| Melting point: | 129-133° C. |

The filtrate is admixed with hydrochloric acid. The precipitate is filtered off using a Nutsch filter, washed and dried.

4′-Trifluoromethylbiphenyl-2-carboxylic acid

| | |
|---|---|
| Yield: | 119.3 g (clear crystals) |
| | 87.4% of the quantity employed |
| Melting point: | 167-170° C. (Lit.: 168-169° C. |

1. A process for preparing a compound of the formula (I): 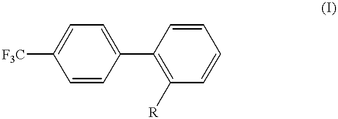
in which R is methyl or carboxyl, which comprises coupling an ortho-tolylmetallate of the formula (II) with a compound of the formula (III) 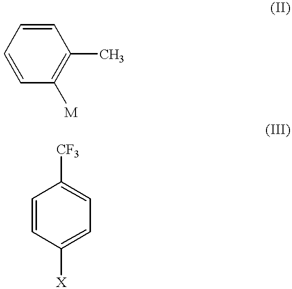
in which
M is —MgF, —MgCl, —MgBr, —Mgl, —Li, —ZnF, —ZnCl, —ZnBr or —Znl and
X is F, Cl, Br, I, N₂⁺, straight-chain or branched (C₁-C₂₀)-alkoxy, arylsulfonate or alkylsulfonate
in the presence of an Ni, Pd or platinum metal catalyst to give a compound of the formula (I) where R is CH₃ and, if appropriate, oxidizing the compound of the formula (I) where R is CH₃ to give the compound of the formula (I) where R is carboxyl.

2. The process as claimed in claim 1, wherein the o-tolylmetallate is o-tolylmagnesium chloride or o-tolylmagnesium bromide.

3. The process as claimed in claim 1, wherein X is Cl, Br, triflate, tosylate, nonaflate or mesylate.

4. The process as claimed in claim 1, wherein the catalyst is PdCl₂(dppf), PdCl₂(dppp), PdCl₂(dppe), PdCl₂(dppb), PdCl₂(PPh₃)₂, Pd(PPh₃)₄, Pd(OAc)₂, PdCl₂, PdBr₂ or NiCl₂(PPh₃)₂.

5. The process as claimed in claim 1, wherein the catalyst is employed in an amount of from 10⁻⁵ to 50 mol %, preferably from 10⁻⁴ to 10 mol %, based on one mole of the compound of the formula (III).

6. The process as claimed in claim 1, wherein the coupling is carried out in an ether, hydrocarbon, halogenated hydrocarbon or a mixture thereof, preferably in THF or THF/toluene mixtures.

7. The process as claimed in claim 1, wherein the coupling is carried out at a temperature of from 0 to 180° C., preferably from 30 to 150° C.

8. The process as claimed in claim 1, wherein the oxidation is carried out using nitric acid, a permanganate, a chromium(VI) compound, oxygen or air.

9. The process as claimed in claim 1, wherein the oxidation is carried out using potassium permanganate in the presence of a phase-transfer catalyst.

10. The process as claimed in claim 1, wherein the oxidation is carried out using air in the presence of an aliphatic carboxylic acid and a heavy metal salt.
